(19) United States
(12) Patent Application Publication
(10) Pub. No.: US 2026/0284044 A1
(43) Pub. Date: Sep. 24, 2026

(54) PHARMACEUTICAL COMPOSITIONS OF VARENICLINE AND PROCESS THEREOF

(71) Applicant: TENSHI KAIZEN L PRIVATE LIMITED, Bangalore, Karnataka (IN)

(72) Inventors: Sathurappan BALAJI, Bangalore, Karnataka (IN); Venkat IYER, Bangalore, Karnataka (IN)

(21) Appl. No.: 19/476,398

(22) PCT Filed: Apr. 20, 2024
(86) PCT No.: PCT/IN2024/050417

§ 371 Date: Oct. 17, 2025

(30) Foreign Application Priority Data
Apr. 20, 2023 (IN) 202341028741

(51) Int. Cl. A61K 31/4995; A61K 9/20

(52) U.S. Cl. CPC A61K 31/4995; A61K 9/2027; A61K 9/2095

(57) ABSTRACT

The present invention discloses a pharmaceutical composition comprising of Varenicline or its pharmaceutically acceptable salts which is substantially free of nitrosamine impurity. More particularly the present invention discloses the pharmaceutical composition of Varenicline or its pharmaceutically acceptable salts and ion-exchange resin. Furthermore, the present invention discloses a stable freeze-dried rapidly disintegrating tablet of Varenicline or its pharmaceutically acceptable salts prepared through lyophilisation process or sublimation techniques.

### CROSS REFERENCE TO RELATED APPLICATIONS

This application claims priority to co-pending Indian Provisional Patent Application Serial Number 202321027324 filed on 20^{th} Apr. 2023. This application is incorporated herein by reference, in its entirety.

### TECHNICAL FIELD OF THE INVENTION

The present invention relates to pharmaceutical composition comprising of Varenicline or its pharmaceutically acceptable salts which is substantially free of nitrosamine impurities. More particularly the present invention relates to the pharmaceutical composition of Varenicline or its pharmaceutically acceptable salts and ion-exchange resin. Furthermore, the present invention relates to a stable freeze-dried rapidly disintegrating tablet of Varenicline or its pharmaceutically acceptable salts prepared through lyophilisation or freeze-dried technology.

### BACKGROUND OF THE INVENTION

Varenicline Tartrate is known as (2R,3R)-2,3-dihydroxybutanedioic acid;5,8,14-triazatetracyclo [10.3.1.02,11.04,9]hexadeca-2,4,6,8,10-pentaene. It was approved by US FDA on May 10, 2006 and marketed by Pfizer under the brand name “CHANTIX”. Varenicline Tartrate is a benzazepine derivative that functions as an ALPHA4-BETA2 NICOTINIC RECEPTOR partial agonist. It has a role as a nicotinic acetylcholine receptor agonist and a serotonergic agonist. Chantix is a treatment to help patients quit smoking and is intended for short term use.

U.S. Pat. No. 6,890,927B2 and U.S. Pat. No. 7,265,119B2 discloses and claims specifically L-tartrate salt of Varenicline and the various polymorphs of the L-tartrate salt, including two distinct anhydrous polymorphs (referred as Forms A and B) and a hydrate polymorph (referred as Form C). Also, it discloses and claims D, L-tartrate salt thereof and its polymorphs, and the meso-tartrate salt thereof and its polymorphs.

US20060057207A1 discloses and claims the different dosages forms of Varenicline and use of Varenicline for treatment of different disorders. It also discloses various excipients used for preparing the dosage forms.

US20130101630A1 discloses methyl varenicline impurity of varenicline, methylvarenicline, 6-methyl-5,8,14-triazatetracyclo[10.3.1.0.sup.2,11,0.sup.4,9]hexadeca-2(1-1),3,5,7,9-pentaene, and a process for the preparation and isolation thereof. Provided further herein is a highly pure varenicline or a pharmaceutically acceptable salt thereof substantially free of methylvarenicline impurity, processes for the preparation thereof, and pharmaceutical compositions comprising highly pure varenicline or a pharmaceutically acceptable salt thereof substantially free of methylvarenicline impurity.

US20200276113A1 discloses and claims the method to mask the bitterness of Varenicline or a pharmaceutically acceptable salt thereof.

In July 2021, Pfizer voluntarily recalled all lots of Chantix 0.5 mg & 1 mg tablets from market as the nitrosamine impurity levels were above the FDA's acceptable intake limits. The FDA permitted varenicline tablets containing N-nitroso-varenicline up to the interim acceptable intake limit of 185ng per day to be distributed in the US in order to maintain an adequate supply of the smoking cessation therapy in the country. The FDA determined that intake limit of 37 nanograms of N-nitroso-varenicline per day is considered reasonably safe for humans, based on lifetime exposure.

N-nitroso-varenicline may increase the risk of cancer if people are exposed to it above the acceptable intake limit and over a long period of time but poses minimal risk to a person taking a drug that contains N-nitroso-varenicline at-or-below the acceptable intake limit every day for 70 years. Agency scientists evaluated the risk of exposure to N-nitroso-varenicline at interim acceptable intake levels up to 185ng per day (92.5 ppm) and determined that it presents minimal additional cancer risk when compared to a lifetime of exposure to N-nitroso-varenicline at the 37 ng per day (18.5 ppm) level.

Different nitrosamines (or more precisely, their reactive metabolites) among the genotoxic N-nitrosamines can cause various kinds of DNA lesions. Nitrosamine impurities may increase the risk of cancer if people are exposed to them above acceptable levels over long periods of time. Further research is necessary to determine the risk of cancer following exposure to potentially contaminated medicinal items since N-nitrosamine contamination in pharmaceuticals is becoming more widely reported, and because N-nitrosamines are powerful mutagenic carcinogens.

There can be many causes for formation of nitrosamine impurities. One of the causes can be due to use of secondary, tertiary, or quaternary amines and nitrite salts under acidic reaction conditions. Nitrite salts form nitrous acid and nitrous acid reacts with amine to form nitrosamine impurity. There is a greater risk of nitrosamine formation if nitrous acid is used to quench residual azide in the presence of precursor amines.

Second reason can be API, intermediates or starting materials that contain secondary or tertiary amine or use of tertiary and quaternary amine as reagents or catalysts which react with nitrous acids or nitrosating agents to form nitrosamines. Other reasons may be contamination of raw materials during shipments or inadequate cleaning of equipment, manufacturing of same API with different synthetic processes and if any, of those process produces nitrosamines or contain precursor amine, contamination of recovered solvents, reagents and catalyst with nitrosamines or lack of optimization of reaction parameters (temp, pH, etc.) during synthesis of APIs.

Nitrosamine impurities may be introduced in finished products through active pharmaceutical ingredients (APIs) or excipients being used. Nitrosamine impurities can be formed by a nitrosating reaction between amines and nitrous acid (nitrite salts under acidic condition). Hence if the API is kept in the vicinity of alkaline or basic excipients, formation of nitrosamine impurity can be avoided.

USFDA has identified seven nitrosamine impurities that theoretically could be present in drug products: N-nitrosodimethylamine (NDMA), N-nitrosodiethylamine (NDEA), N-nitroso-N-methyl-4-aminobutanoic acid (NMBA), N-nitrosoisopropylethyl amine (NIPEA), N-nitrosodiisopropylamine (NDIPA), N-nitrosodibutylamine (NDBA), and N-nitrosomethylphenylamine (NMPA). Five of them (NDMA, NDEA, NMBA, NIPEA, and NMPA) have actually been detected in drug substances or drug products.

The supplier qualification program should consider that nitrite impurities vary across excipient lots and may vary by supplier. Nitrite and nitrosamine impurities may be present in potable water. The risk assessment should also include evaluation of any pathway (including degradation) that may introduce nitrosamines during drug product manufacture or storage.

There is therefore, a need for providing pharmaceutical composition of varenicline or a pharmaceutically acceptable salt which is substantially free of N-nitroso varenicline impurity and the process thereof.

### SUMMARY OF THE INVENTION

The primary object of the present invention is to provide a pharmaceutical composition comprising of Varenicline or its pharmaceutically acceptable salts substantially free from nitrosamine impurity and the process for preparation thereof.

The other object of the invention is to provide a stable freeze-dried rapidly disintegrating tablet of Varenicline or its pharmaceutically acceptable salts prepared through lyophilisation process or sublimation techniques.

In an aspect, the present invention provides a pharmaceutical composition comprising;
(i) Varenicline or its pharmaceutically acceptable salts as the active ingredient;
(ii) ion-exchange resin;
together with pharmaceutically acceptable excipients;
wherein, said pharmaceutical composition is substantially free from nitrosamine impurity.

In another aspect, the ion-exchange resin in the composition/formulation is selected from polyacryls such as a cross linked polymer of acrylic acid and divinylbenzene, a crosslinked polymer of methacrylic acid and divinylbenzene and aminoalkyl methacrylate copolymers; wherein said ion-exchange resin is used to control the formation of nitrosamine impurity in said Varenicline composition.

The pharmaceutically acceptable excipients are selected from the group comprising a plasticizer, a disintegrant, a diluent, a solvent, a penetration enhancer, a preservative, a buffer, a gelator, a lubricant, a carrier, a stabilizer, a gel, a dye, a pigment, a surfactant, an inert filler, an adhesive, a texturizer, a softener, an emulsifier, a sweetener, a flavor and mixtures thereof.

In another aspect, the present invention provides stable freeze-dried rapidly disintegrating pharmaceutical composition of Varenicline or its pharmaceutically acceptable salts which is substantially free from nitrosamine impurity.

In yet another aspect, the present invention provides a process for formulating the pharmaceutical composition of Varenicline or its pharmaceutically acceptable salts which is substantially free from nitrosamine impurity.

In an aspect, the present invention provides a lyophilized process for formulating the pharmaceutical composition of Varenicline or its pharmaceutically acceptable salts.

Accordingly, the process for preparation of Varenicline or its pharmaceutically acceptable salts substantially free from nitrosamine impurity comprising;
(i) Dissolving ion-exchange resin in water to obtain the solution;
(ii) Adding Varenicline or its pharmaceutically acceptable salt to the solution of step (i) followed by addition of suitable pharmaceutical excipients;
(iii) Homogenizing the suspension of step (ii), filling the same into blister cavities and freezing to obtain the desired composition; and
(iv) Loading the frozen composition of step (iii) and drying.

The inert solid ingredients are selected from urea, ammonium bicarbonate, ammonium carbonate, hexamethylene tetramine, naphthalene, phthalic anhydride, benzoic acid, camphor, menthol alone or mixtures thereof.

The excipients are selected from the group consisting of additive selected from but, not limited to, excipient, a plasticizer, a disintegrant, a diluent, a solvent, a penetration enhancer, a preservative, a buffer, a gelator, a lubricant, a carrier, a stabilizer, a gel, a dye, a pigment, a surfactant, an inert filler, an adhesive, a texturizer, a softener, an emulsifier and mixtures thereof as commonly known in the pharmaceutical field.

### DETAILED DESCRIPTION OF THE INVENTION

The present invention will now be explained in detail with reference to its preferred and optional embodiments but should not be construed to limit the scope of the invention.

Nitrites are common nitrosating impurities that have been reported in many excipients at ppm levels. Nitrite impurities are found in a range of commonly used excipients, which may lead to nitrosamine impurities forming in drug products during the drug product manufacturing process and shelf-life storage period.

Most probable cause might be PEG used in coating material. Oxidation of PEGs at high temperature at a central carbon followed by chain scission could generate formaldehyde. Air oxidation of formaldehyde at temperatures used for accelerated stability testing could lead to the formation of formic acid, which would then react with alcohols to form esters. Formic acid could also be formed through the oxidation of terminal groups in PEG and polysorbates. Formic acid can cause drug instability in the formulations. For example, Waterman et al. observed N-formylation and N-methylation of the secondary amine group in varenicline. These were attributed to the presence of formaldehyde and formic acid in the formulation. N-methyl varenicline (NMV) and N-formyl varenicline (NFV) were formed in the osmotic tablets of varenicline, a secondary amine. NMV was produced by the reaction of the amine moiety with both formaldehyde and formic acid impurities in an Eschweiler-Clarke reaction, while NFV was formed by the reaction of formic acid alone with varenicline. Other likely causes for n-nitrosamine generation are solvents used in the process, purified water (Presence of residual dialkyl amines or impurities that can degrade to form dialkyl amines and presence of nitrites or other nitrosating agents).

For the purpose of the present invention, the term ‘composition’ and ‘formulation’ can be used interchangeably having the same meaning.

Similarly, the term ‘N-nitroso impurity’ and ‘nitroso impurity’ can be used interchangeably having the same meaning.

The pharmaceutical composition of Varenicline or its pharmaceutically acceptable salts along with ion-exchange resin and pharmaceutically acceptable excipients substantially free from nitrosamine impurity would mean pharmaceutical composition of Varenicline or its pharmaceutically acceptable salts along with ion-exchange resin and pharmaceutically acceptable excipients wherein the nitrosamine impurity is present in an amount ranging from 1 ppm to 2 ppm.

The present invention is directed to providing pharmaceutical composition of Varenicline, with reduced levels of nitrosamine impurity hitherto not achieved in the art.

In an embodiment, the present invention discloses a pharmaceutical composition comprising;
(i) Varenicline or its pharmaceutically acceptable salts as the active ingredient;
(ii) ion-exchange resin;
together with pharmaceutically acceptable excipients;
wherein, said pharmaceutical composition is substantially free from nitrosamine impurity.

In another embodiment, the nitrosamine impurity in the present pharmaceutical composition is present in an amount less than 2 ppm.

In another embodiment, the nitrosamine impurity in the present pharmaceutical composition is present in an amount ranging from 1 ppm to 2 ppm.

The Varenicline or its pharmaceutically acceptable salts as the active ingredient in the composition is present in an amount ranging between 0.1-1.5% w/w.

In an embodiment, the ion-exchange resin in the composition/formulation is selected from polyacryls such as selected from group consisting of crosslinked polymer of acrylic acid and divinylbenzene, a crosslinked polymer of methacrylic acid and divinylbenzene and aminoalkyl methacrylate copolymers. The non-limiting examples include EudragitE100®, EudragitEPO® and EudragitE12.5®, styrene-based copolymers such as Kyron T-123®, Indion454®, Indion204® and acrylic acid copolymers such as Purolite A830EMR®,etc. and natural cationic polymers such as lecithin, chitosan, gelatin, etc.; wherein said ion-exchange resin is used to control the formation of nitrosamine impurity in said Varenicline composition. The ion-exchange resin is present in the composition in an amount ranging between 2.0 to 8% w/w.

In an embodiment, a complex could be formed between the Varenicline drug or its pharmaceutically acceptable salts and ion-exchange resin wherein, the carboxylic functional group of the resin will form a complex with the free —NH group of Varenicline. The nature of the complex is such that the compositions of Varenicline are substantially free of N nitroso impurity.

The nature of the complex is such that the average cation concentration of about 40 meq/1 and pH of 6.7 in saliva is not able to break the complex. However, the complex is weak enough to be broken down by the hydrochloric acid present in the stomach. Thus, the complex formed is absolutely tasteless and stable with no after taste, but at the same time, its bioavailability is not affected.

Surprisingly, the present inventors found that the composition comprising ion-exchange resin was stable and did not form N-nitroso impurities even after storing for 6 months at temperature as high as 40° C. On the other hand, the composition comprising beta cyclodextrin which was prepared using lyophilization process had about 3 ppm of N-nitroso impurities immediately after preparation. Further the composition prepared by batch compression technique had approximately 13 ppm of impurity as soon as it was prepared. The novelty and inventive aspect of present invention resides in the composition and process to prepare said composition, wherein the composition comprises varenicline or its pharmaceutically acceptable salt and an ion-exchange resin. Further inventive aspect also lies in the stable composition of varenicline that is free from N-nitroso impurities when studied over a period of at least 6 months. The prior art does not disclose or teach a composition comprising varenicline or its pharmaceutically acceptable salt and an ion-exchange resin let alone the stable composition devoid of N-nitroso impurities.

Further it should be noted that even though, the ion-exchange resin aids in controlling/formation of nitrosamine impurity in the Varenicline compositions, is non-toxic and does not negatively affect the pharmacological activity of Varenicline or its pharmaceutically acceptable salt.

The pharmaceutically acceptable carriers are selected from the group consisting of additive selected from but, not limited to, excipient, a plasticizer, a disintegrant, a diluent, a solvent, a penetration enhancer, a preservative, a buffer, a gelator, a lubricant, a carrier, a stabilizer, a gel, a dye, a pigment, a surfactant, an inert filler, an adhesive, a texturizer, a softener, an emulsifier, flavors, sweeteners; and the like or mixtures thereof.

Varenicline is potentially known for its bitter taste and therefore, it requires a suitable taste masking agent to enhance the mouth feels.

The flavors selected are derived from natural and synthetic sources such as volatile oils, synthetic flavor oils, flavoring aromatics, oils, liquids, oleoresins or extracts derived from plants, leaves, flowers, fruits, stems and the like. The examples of flavors include but are not limited to, cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil, citrus oils such as lemon, orange, grape, lime and grapefruit and fruit essences such as apple, pear, peach, grape, strawberry, raspberry, cherry, plum, pineapple, apricot, and other fruit flavors. Preferably, the flavor used is the present invention is peppermint flavor.

The sweeteners are selected from, but not limited to, corn syrup, dextrose, invert sugar, fructose, and mixtures thereof; saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, xylitol; and the like. Preferably, the sweetener used in the present invention is dipeptide sweetener such as Aspartame.

The diluents are selected from, but not limited to, mannitol, sorbitol, xylitol, microcrystalline cellulose (e.g., Avicel® PH200, PH102 or PH101 available from FMC BioPolymer, Philadelphia, Pa.), silicified microcrystalline cellulose (such as Prosolv® available from PenWest Pharmaceuticals Co., Patterson, N.Y.), cellulosic polymers such as hydroxypropyl methylcellulose (HPMC) and hydroxypropyl cellulose (HPC), pullulan and fast dissolving carbohydrates (such as Pharmaburst™ available from SPI Pharma, New Castle, Del.).

The glidants are selected from, but not limited to, silicon dioxide, colloidal silicon dioxide, calcium silicate, magnesium silicate, magnesium trisilicate, talc, starch, combinations thereof, and the like.

In another aspect, the present invention provides stable freeze-dried rapidly disintegrating pharmaceutical composition of Varenicline or its pharmaceutically acceptable salts which is substantially free from nitrosamine impurity.

In an embodiment, the pharmaceutical composition of Varenicline or its pharmaceutically acceptable salts which is substantially free from nitrosamine impurity may be prepared through lyophilization or sublimation process.

Accordingly, the present invention provides a lyophilized process for formulating the pharmaceutical composition of Varenicline or its pharmaceutically acceptable salts.

The lyophilisation process for preparation of Varenicline or its pharmaceutically acceptable salts substantially free from nitrosamine impurity comprising;
(i) Dissolving ion-exchange resin in water to obtain the solution;
(ii) Adding Varenicline or its pharmaceutically acceptable salt to the solution of step (i) followed by addition of suitable pharmaceutical excipients;
(iii) Homogenizing the suspension of step (ii), filling the same into blister cavities and freezing to obtain the desired composition; and
(iv) Loading the frozen composition of step (iii) and drying.

In an embodiment, the film forming polymer may be added to the process step (ii) of the present invention, wherein, the polymer is selected from pullulan, hydroxypropyl cellulose (HPC), hydroxypropyl methylcellulose (HPMC), polyvinylpyrrolidone (PVP), starch, a polyethylene glycol polyvinyl alcohol copolymer, copovidone, hydroxyethyl cellulose, hydroxypropyl starch, polyethylene oxide, poloxamer or a mixture thereof.

In yet another embodiment, the present invention discloses a process for preparation of Varenicline or its pharmaceutically acceptable salt by sublimation method. The sublimation technique used provides highly porous compressed tablets.

The inert solid ingredients are selected from urea, ammonium bicarbonate, ammonium carbonate, hexamethylene tetramine, naphthalene, phthalic anhydride, benzoic acid, camphor, menthol.

The excipients are selected from the group consisting of additive selected from but, not limited to, excipient, a plasticizer, a disintegrant, a diluent, a solvent, a penetration enhancer, a preservative, a buffer, a gelator, a lubricant, a carrier, a stabilizer, a gel, a dye, a pigment, a surfactant, an inert filler, an adhesive, a texturizer, a softener, an emulsifier and mixtures thereof as commonly known in the pharmaceutical field. The said pharmaceutically acceptable excipients/carrier in the composition is in an amount ranging between 0.01 to 8% w/w.

In an embodiment, the present invention discloses pharmaceutical composition of Varenicline or its pharmaceutically acceptable salts substantially free of N-nitroso impurity for use as Alpha4-Beta2 Nicotinic receptor partial agonist.

In yet another embodiment, the present invention discloses the pharmaceutical composition to quit/discontinue smoking.

In another embodiment, the present invention provides a method of smoking cessation comprising providing the pharmaceutical composition of Varenicline or its pharmaceutically acceptable salts substantially free of N-nitroso impurity.

The following examples, which include preferred embodiments, will serve to illustrate the practice of this invention, it being understood that the particulars shown are by way of examples and for purpose of illustrative discussion of preferred embodiments of the invention.

### EXAMPLES

### General Composition of the Present Invention

| Ingredients | % Weight |
|---|---|
| Varenicline Tartrate | 0.1-1.5% w/w |
| Ion-exchange resin | 2.0 to 8% w/w |
| Pharmaceutically acceptable Excipients | 0.01 to 8% w/w |

### Example 1: Composition with Indion 204 (Cation Exchange Resin)

| | Strength | |
|---|---|---|
| | 0.5 mg | 1 mg |
| Ingredients | % Weight | % Weight |
| Varenicline Tartrate | 0.43 | 0.43 |
| Pullulan | 2.00 | 2.00 |
| Mannitol 25 C | 7.50 | 7.50 |
| Indion 204 | 4.28 | 4.28 |
| Aspartame | 1.00 | 1.00 |
| Mint | 0.50 | 0.50 |
| Purified water | 84.30 | 84.30 |
| Total | 100.00 | 100.00 |

### Manufacturing Process:

Step 1: Required quantity of purified water was taken in a 250 ml glass beaker.
Step 2: Required quantity of Indion 204 was added to step 1 under stirring using mechanical stirrer and continued mixing for 5 mins at 400 rpm.
Step 3: Required quantity of varenicline was added to step 2 and continued mixing for 5 mins at 400 rpm.
Step 4: Required quantity of pullulan was added to step 3 and continued mixing for 5 mins at 400 rpm.
Step 5: Required quantity of mannitol 25C was added to step 4 and continued mixing for 5 mins at 400 rpm.
Step 6: Required quantity of aspartame was added to step 5 and continued mixing for 1 hour at 400 rpm.
Step 7: Required quantity of peppermint flavour was added to step 6 and continued mixing for 5 mins at 400 rpm.
Step 8: The suspension was homogenized @4000 rpm for 10 mins.
Step 9: The suspension of step 8 was filled into preformed 0.45 ml blister cavities and subjected to quick freezing at −90° C. using liquid nitrogen.
Step 10: Frozen tablets were loaded into lyophilizer and dried as per the given lyo cycle parameters i.e. drying temperature ranging from −30° C. to 0° C. followed by lyophilizing at a secondary drying temperature ranging from 15° C. to 40° C.
Step 11: After completion of drying cycle the tablets were unloaded and sealed.

TABLE 1

| Sr. | | | | |
|---|---|---|---|---|
| No | Name of sample | Batch No. | Results | Limit |
| 1 | Varenicline API | API (EL-01-A179- | Less than 2 ppm | NMT |
| | | 2001003) | | 18.5 |
| 2 | Varenicline Tablets | 001 (initial) | Less than 2 ppm | ppm |
| | 1 mg | | | |
| 3 | Varenicline Tablets | 001 (1M; | Less than 2 ppm | |
| | 1 mg | 40° C./75% RH) | | |
| 4 | Varenicline Tablets | 002 (Initial) | Less than 2 ppm | |
| | 1 mg | | | |

Varenicline tablets were manufactured using Lyophilization technology, using excipients that do not pose the risk of forming Nitrosamines during processing and during product shelf life. The process involves filling of a suspension of the drug along with suitable excipients into preformed blisters that are subjected to process of quick freezing followed by Lyophilization and sealing. In Lyophilization technique, the water is removed from the product after it gets frozen, allowing the ice to change directly from solid to vapor without passing through liquid phase. The process leaves behind a porous matrix in the shape of a tablet, which disintegrates quickly in less than 10 seconds. The manufacturing process does not employ temperatures exceeding 25° C. The short-term stability data shows levels of nitroso varenicline well below the prescribed limits by the FDA.

### Example 2: Comparative Composition with Cyclodextrin

| | Strength |
|---|---|
| | 1 mg |
| Ingredients | % Weight |
| Varenicline Tartrate | 0.43 |
| Pullulan | 2.00 |
| Mannitol | 7.50 |
| Beta cyclodextrin | 4.28 |
| Aspartame | 1.00 |
| Mint | 0.50 |
| Purified water | 84.30 |
| Total | 100.00 |

### Manufacturing Process:

Step 1: Required quantity of purified water was taken in a 250 ml glass beaker.
Step 2: Required quantity of beta cyclodextrin was added to step 1 under stirring using mechanical stirrer and continued mixing for 5 mins at 400 rpm.
Step 3: Required quantity of varenicline was added to step 2 and continued mixing for 5 mins at 400 rpm.
Step 4: Required quantity of pullulan was added to step 3 and continued mixing for 5 mins at 400 rpm.
Step 5: Required quantity of mannitol was added to step 4 and continued mixing for 5 mins at 400 rpm.
Step 6: Required quantity of aspartame was added to step 5 and continued mixing for 1 hour at 400 rpm.
Step 7: Required quantity of peppermint flavour was added to step 6 and continued mixing for 5 mins at 400 rpm.
Step 8: The suspension was homogenized @4000 rpm for 10 mins.
Step 9: The suspension of step 8 was filled into preformed 0.45 ml blister cavities and subjected to quick freezing at −90° C. using liquid nitrogen.
Step 10: Frozen tablets were loaded into lyophilizer and dried as per the given lyo cycle parameters.
Step 11: After completion of drying cycle the tablets were unloaded and sealed.

TABLE 2

| Sr. | | | |
|---|---|---|---|
| No | Name of sample | Results | Limit |
| 1 | Varenicline Tablets 1 mg | 3 | 18.5 ppm |
| | (with beta cyclodextrin) | ppm | |
| | | (Initial) | |

### Example 3: Comparative Compression Batch Composition (Chantix)

| | Strength |
|---|---|
| | 1 mg |
| Ingredients | % Weight |
| Varenicline Tartrate | 1.14 |
| Microcrystalline cellulose | 79.19 |
| Dibasic calcium phosphate | 12.67 |
| Croscarmellose sodium | 2.00 |
| Polyvinyl pyrrolidone (Povidone k-30) | 4.00 |
| Colloidal silicon dioxide | 0.50 |
| Magnesium stearate | 0.50 |
| Purified water* | q.s. |
| Total | 100.00 |

*removed during the manufacturing process

### Manufacturing Process:

Step 1: Dispensed all the materials as per manufacturing formula.
Step 2: Required quantity of Microcrystalline cellulose was added into a bowl.
Step 3: Required quantity of Dibasic calcium phosphate was added to the step 2 and mixed it manually by hand.
Step 4: Required quantity of Croscarmellose sodium was added to the step 3 and mixed it manually by hand.
Step 5: Required quantity of Povidone k-30 was added to the step 4 and mixed it manually by hand.
Step 6: Required quantity of Purified water was taken in a beaker and dispensed quantity of Varenicline was added in it and mixed manually to form a clear solution.
Step 7: Solution of step 6 was added into step 5 and mixed manually by hand until proper granules formed.
Step 8: After hand granulation dry the granules for 10 minutes in hot air oven at 40° C. and collected it in a polybag.
Step 9: Required quantity of Colloidal silicon dioxide was added to the dried granules of step 8 and mixed it manually in a polybag for 3 minutes.
Step 10: Required quantity of Magnesium stearate was added to the step 9 and mixed it manually in a polybag for 2 minutes.
Step 11: Step 10 blend was weighed 150 mg individually and compressed manually in SRC10i Compression machine by using 8 mm punches.

The n-nitroso varenicline content of the initial sample is given below.

| S. No | Sample details | Results | Limit |
|---|---|---|---|
| 1 | Initial | 13.75 ppm | 18.5 ppm |

Varenicline Orally disintegrating tablets 1 mg N-nitroso varenicline results:
Initial data:

| S. | | Initial (ppm) |
|---|---|---|
| No. | Product details | (N-nitroso varenicline) |
| 1 | With Indion 204 resin and | 0.13 |
| | lyophilization process | |
| 2 | With beta cyclodextrin and | 3.02 |
| | lyophilization process | |
| 3 | With amberlite IRP 64 resin | 1.72 |
| 4 | Compressed tablets | 13.75 |
| | (conventional wet granulation | |
| | process) | |
| 5 | Varenicline tartrate (API) | 1.73 |

1. Batch No. VAR/T/1/001

| | | | | | 40° C./75% RH | | |
|---|---|---|---|---|---|---|---|
| S. | Product | Batch | Mfg. | Initial | 3 | | |
| No | name | No. | date | (ppm) | weeks | 3M | 6M |
| 1 | Varenicline | VAR/T/ | Apr. 22, 2022 | 1.55 | 1.62 | 2.58 | 1.93 |
| | tablets 1 mg | 1/001 | | (1M-RT) | ppm | ppm | ppm |

2. Batch No. VAR/T/1/002

| S. | Product | | Mfg. | Initial | 25° C./60% RH | | | 40° C./75% RH | | |
|---|---|---|---|---|---|---|---|---|---|---|
| No. | name | Batch No. | date | (ppm) | 1M | 2M | 3M | 1M | 2M | 3M |
| 1 | Varenicline | VAR/T/1/002 | May 19, 2022 | 0.13 | 0.29 | 0.14 | 0.16 | 0.17 | 0.15 | 0.93 |
| | tablets | | | ppm | ppm | ppm | ppm | ppm | ppm | ppm |
| | 1 mg | | | | | | | | | |

### Advantages

(i) As Lyophilized process was used, it avoided heating step (temperature about 25° C. which generally gives rise to Nitrosamine impurities.
(ii) It was found that the compressed tablets n-nitroso varenicline content was 13.75 ppm against less than 2 ppm in lyophilized tablets containing ion exchange resin (methacrylic acid divinyl benzene and acrylic acid i.e. Indion 204).
(iii) After comparing the 6 months data at room temperature and elevated temperature, it is clear that the N-nitroso varenicline content in Varenicline tablets is less than 2 ppm.

Examples as above are not limiting the scope of the invention. The quantities of the ingredients and the process steps thereof may be modified suitably to meet the lyophilizing or freeze-drying parameters and the consistency of the final suspension or solution being filled into the blister cavities for freeze drying and sealing.

1. A pharmaceutical composition comprising:
(i) varenicline or a pharmaceutical acceptable salt thereof;
(ii) an ion exchange resin; and
(iii) a pharmaceutically acceptable excipient;
wherein the pharmaceutical composition is substantially free of a nitrosamine impurity.

2. The pharmaceutical composition according to claim 1, wherein varenicline or the pharmaceutical acceptable salt thereof is in a concentration ranging from 0.1% w/w to 1.5% w/w.

3. The pharmaceutical composition according to claim 1, wherein the ion-exchange resin comprises a crosslinked polymer of acrylic acid and divinylbenzene, a crosslinked polymer of methacrylic acid and divinylbenzene, or an aminoalkyl methacrylate copolymers.

4. The pharmaceutical composition according to claim 3, wherein the ion exchange resin is in a concentration ranging from 2% w/w to 8% w/w.

5. The pharmaceutical composition according to claim 1, wherein the pharmaceutically acceptable excipient comprise a plasticizer, a disintegrant, a diluent, a solvent, a penetration enhancer, a preservative, a buffer, a gelator, a lubricant, a carrier, a stabilizer, a gel, a dye, a pigment, a surfactant, an inert filler, an adhesive, a texturizer, a softener, an emulsifier, a sweetener, a flavor, or a mixtures thereof in a concentration ranging from 0.01% w/w to 8% w/w.

6. The pharmaceutical composition according to claim 5, wherein the inert filler comprises urea, ammonium bicarbonate, ammonium carbonate, hexamethylene tetramine, naphthalene, phthalic anhydride, benzoic acid, camphor, menthol alone, or a mixture thereof.

7. The pharmaceutical composition according to claim 1, wherein the pharmaceutical composition is a stable freeze-dried rapidly disintegrating tablet.

8. The pharmaceutical composition according to claim 1, wherein the nitrosamine impurity is in an amount ranging from 1 ppm to 2 ppm.

9. A stable freeze-dried rapidly disintegrating tablet comprising varenicline or a pharmaceutically acceptable salt thereof and an ion exchange resin substantially free of a nitrosamine impurity.

10. The stable freeze-dried rapidly disintegrating tablet according to claim 9, wherein the nitrosamine impurity is in an amount ranging from 1 ppm to 2 ppm.

11. A process for the preparation of the stable freeze-dried rapidly disintegrating tablet according to claim 9, the process comprising:
(i) dissolving the ion exchange resin in water to obtain a solution;
(ii) adding varenicline or the pharmaceutically acceptable salt thereof to the solution of step (i) followed by addition of a pharmaceutically acceptable excipient to form a suspension;
(iii) homogenizing the suspension of step (ii) to form a homogenised suspension;
(iv) filling the homogenised suspension of step (iii) into blister cavities and quick freezing at −90° C. to obtain a frozen rapidly disintegrating tablet; and
(v) loading the frozen rapidly disintegrating tablet of step (iv) and drying at a primary drying temperature ranging from −30° C. to 0° C. followed by lyophilizing at a secondary drying temperature ranging from 15° C. to 40° C. to obtain the stable freeze-dried rapidly disintegrating tablet.

12. (canceled)

13. (canceled)

14. A method of smoking cessation, the method comprising providing the pharmaceutical composition as claimed in claim 1.

15. The process of claim 11, wherein the homogenizing is conducted at 400 rpm.

16. The pharmaceutical composition according to claim 1, wherein varenicline or the pharmaceutical acceptable salt thereof is in a concentration ranging from 0.1% w/w to 1.5% w/w, the ion exchange resin is in a concentration ranging from 2% w/w to 8% w/w, and the pharmaceutically acceptable excipient is in a concentration ranging from 0.01% w/w to 8% w/w.

17. The pharmaceutical composition according to claim 1, wherein a complex is formed between varenicline or the pharmaceutical acceptable salt thereof and the ion exchange resin.

18. The pharmaceutical composition according to claim 16, wherein a complex is formed between varenicline or the pharmaceutical acceptable salt thereof and the ion exchange resin.
